(19) United States
(12) Patent Application Publication
(10) Pub. No.: US 2004/0199056 A1
(43) Pub. Date: Oct. 7, 2004

(54) Body monitoring using local area wireless interfaces

(75) Inventors: Dirk Husemann, Adliswil (CH); Robert E. Steinbugler, Raleigh, NC (US); Bryan L. Striemer, Zumbrota, MN (US)

Correspondence Address: Robert R. Williams, IBM Corporation, Dept. 917, 3605 Highway 52 North, Rochester, MN, 55901-7829, US

(73) Assignee: INTERNATIONAL BUSINESS MACHINES CORPORATION, ARMONK, NY

(21) Appl. No.: 10/406,865

(22) Filed: Apr. 3, 2003

(51) Int. Cl. H04M 11/04; A61B 5/00

(52) U.S. Cl. 600/300; 455/404.1; 128/903; 128/904; 455/406

(57) ABSTRACT

Methods and systems for placing a call or sending a message from a mobile telephone in response to receiving a signal from a portable externally worn body relay device or body monitoring device. The portable device and the mobile telephone are equipped with local area wireless interfaces. In one embodiment, the mobile telephone places a distress call to a predefined telephone number.

### BACKGROUND OF THE INVENTION

1. Field of the Invention

The present invention generally relates to telemetry and body monitors. More particularly, the invention relates to methods and systems for transmitting physiological information to a mobile phone or other telemetry device.

2. Description of the Related Art

Telemetry refers to the communication of devices. Telemetry has broad application for transmission of data and control signals, for example. With the increasing shrinking size of chips, more functionality can be provided in increasingly smaller telemetry devices.

One area where telemetry devices are being exploited, is in the medical services community. Systems are now available which can be worn by patients, record vital information regarding a patient's health and transmit the vital information to another device.

However, a number of shortcomings exist with respect to such existing telemetry systems. In some cases, the systems require expensive proprietary equipment uniquely configured to perform medical service functions. The systems may or may not be portable but, in any case, require users to possess and operate function-specific equipment which they would not otherwise use. In addition, many systems are limited to information gathering and do not respond to emergency situations. For example, one real-time monitoring system downloads information from a pacemaker and, ultimately, transmits the downloaded information to a physician. However, the operation of such real-time monitoring systems is not dependent upon, or triggered by, the condition of the patient being monitored. Rather, the downloading and transmitting of information occurs at a preset interval. A determination that the patient requires medical attention can only be made by the doctor after receiving and reviewing the downloaded information. Because the downloaded information does not explicitly notify the physician of a possible emergency condition, the physician is not motivated to take immediate emergency action. As a result, critical time may pass between the time the information is made available for review and the time that the information is actually reviewed.

Therefore, there is a need for telemetry devices capable of medical/health service functions using, in part, existing telecommunications infrastructure.

### SUMMARY OF THE INVENTION

The present invention generally provides methods and systems capable of medical/health service functions using, in part, existing telecommunications infrastructure. In one embodiment, a call is placed from a mobile telephone in response to receiving a signal from a portable externally worn body relay device or body monitoring device. The portable device and the mobile telephone are equipped with local area wireless interfaces. In one embodiment, the mobile telephone places a distress call to a predefined telephone number.

In one aspect, a method for placing a distress call from a mobile telephone in response to detected body functions of a human subject includes receiving, by a local area wireless interface of the mobile telephone, a wireless signal from a corresponding local area wireless interface disposed on an external body-worn device; in response to receiving the wireless signal, dialing a telephone number corresponding to a device; and transmitting, from a wireless telephone communications facility of the mobile telephone, the distress call to the device.

In another aspect, a wireless telephone for selectively placing a telephone call, includes a local area wireless interface configured to receive transmissions from a corresponding local area wireless interface disposed on a body-worn device externally disposed on a human subject; a wireless telephone facility; a processing unit; instructions for configuring the processing unit to respond to transmissions from the body-worn device by initiating the telephone call, via the wireless telephone facility, to a predefined telephone number.

In yet another aspect, an external body-worn relay device includes a receiver configured for wireless communication with a body sensor; a body monitoring device configured to receive signals received from the body sensor via the receiver and monitor an aspect of human activity of a human subject; a local area wireless transmitter in communication with the monitoring device and configured to transmit a wireless signal in response to a local area wireless interface of a mobile telephone.

In yet another aspect, a portable externally worn body monitoring device includes a monitoring device configured to monitor an aspect of human activity of a human subject; a local area wireless transmitter in communication with the monitoring device and configured to transmit a wireless signal to a local area wireless interface of a mobile telephone; and a support member for supporting the monitoring device and the local area wireless transmitter on the human subject.

In yet another aspect, a portable externally worn body monitoring device includes a monitoring device interface configured to interface with a monitoring device monitoring an aspect of human activity of a human subject; a local area wireless transmitter in communication with the monitoring device and configured to transmit a wireless signal to a local area wireless interface of a mobile telephone; and a support member for supporting the monitoring device and the local area wireless transmitter on the human subject.

In still another aspect, a method of providing a distress call service by a mobile telephone service provider includes receiving distress calls from mobile telephones equipped with local area wireless interfaces configured to communicate with corresponding local area wireless interfaces of portable externally worn body monitoring devices, wherein the portable externally worn body monitoring devices cause the mobile telephones to transmit the distress calls by transmitting a signal from their respective corresponding local area wireless interfaces to the local area wireless interfaces of the wireless telephones; connecting the mobile telephones to receiving devices having telephone numbers to which the distress calls are made; transmitting, from the mobile telephone, information indicating an emergency to the receiving devices; and charging a fee for the connecting and transmitting.

In still another aspect, a mobile telephone gathers and timestamps signals from a portable, externally worn body monitoring or relay device and periodically transmits a collection of signals at regular intervals over the mobile telephone's data channel to a system running in the network. In one embodiment, the mobile telephone collects signals from portable, externally worn body monitoring or relay devices and evaluates the received signals and sends an alert message to a back end system or to another phone.

### BRIEF DESCRIPTION OF THE DRAWINGS

So that the manner in which the above recited features of the present invention are attained and can be understood in detail, a more particular description of the invention, briefly summarized above, may be had by reference to the embodiments thereof which are illustrated in the appended drawings.

It is to be noted, however, that the appended drawings illustrate only typical embodiments of this invention and are therefore not to be considered limiting of its scope, for the invention may admit to other equally effective embodiments.

FIG. 1 is a diagram of a human subject wearing a plurality of devices equipped with local area wireless interfaces configured to communicate with an external wireless device having a corresponding wireless interface.

FIG. 2 is a block diagram of one embodiment of a body-worn device in communication with a detector unit and an external wireless device.

FIG. 3 is a block diagram of another embodiment of a body-worn device having a signal processing unit, and in communication with a detector unit and an external wireless device.

FIG. 4 is a block diagram of another embodiment of a body-worn device in communication with an integrated detector unit and an external wireless device.

FIG. 5 is a block diagram of another embodiment of a body-worn device equipped with a locator unit, and in communication with a detector unit and an external wireless device.

### DETAILED DESCRIPTION OF THE PREFERRED EMBODIMENTS

The present invention generally provides methods and systems for using telemetry devices capable of transmitting medical/health information via a mobile telephone network. Generally, the medical/health information may include data or control signals provided by medical/health devices (including medicine dispensers and body monitors). The information is transmitted from telemetry device, worn by a human being, to a telephone network. In one embodiment, the telemetry device worn by a human being is a communication relay between a body sensor and a mobile telephone which, in turn, communicates with the telephone network. In another embodiment, the telemetry device includes a mobile telephone facility and can communicate with the telephone network directly. In another embodiment, the mobile telephone collects signals from the medical/health devices over some period of time and transmits a collection of signals to the telephone network or a server in the Internet. In a particular embodiment, the mobile telephone does a first evaluation of the received signals and may send an (alarm) message to the telephone network or a server in the Internet to trigger a download of the collected sensor data.

In a particular embodiment, the medical/health information is indicative of a medical emergency for a particular human being. The telemetry device worn by the human being communicates with a wireless mobile telephone and causes the wireless mobile telephone to initiate a distress call over a telephone network. Although aspects of the present invention are described with respect to human subjects, the telemetry devices may also be worn by non-human animals.

Some or all aspects of the invention may be implemented as a program product comprising executable code. The program(s) of the program product defines functions of the embodiments (including the methods described below) and can be contained on a variety of signal-bearing media. Illustrative signal-bearing media include, but are not limited to: (i) information permanently stored on non-writable storage media (e.g., read-only memory devices within a computer such as CD-ROM disks readable by a CD-ROM drive); (ii) alterable information stored on writable storage media (e.g., floppy disks within a diskette drive or hard-disk drive); or (iii) information conveyed to a computer by a communications medium, such as through a computer or telephone network, including wireless communications. The latter embodiment specifically includes information downloaded from the Internet and other networks. Such signal-bearing media, when carrying computer-readable instructions that direct the functions of the present invention, represent embodiments of the present invention.

While functions of the invention may be implemented in software, the same functions may also be implemented in hardware. For example, hardwired embedded controllers and Application Specific Integrated Circuits (ASIC) may be used. Accordingly, the invention may be implemented in software, hardware or a combination of both.

In addition, various programs/instructions described hereinafter may be identified based upon the application for which they are implemented in a specific embodiment of the invention. However, it should be appreciated that any particular program/instructions nomenclature that follows is used merely for convenience, and thus the invention should not be limited to use solely in any specific application identified and/or implied by such nomenclature.

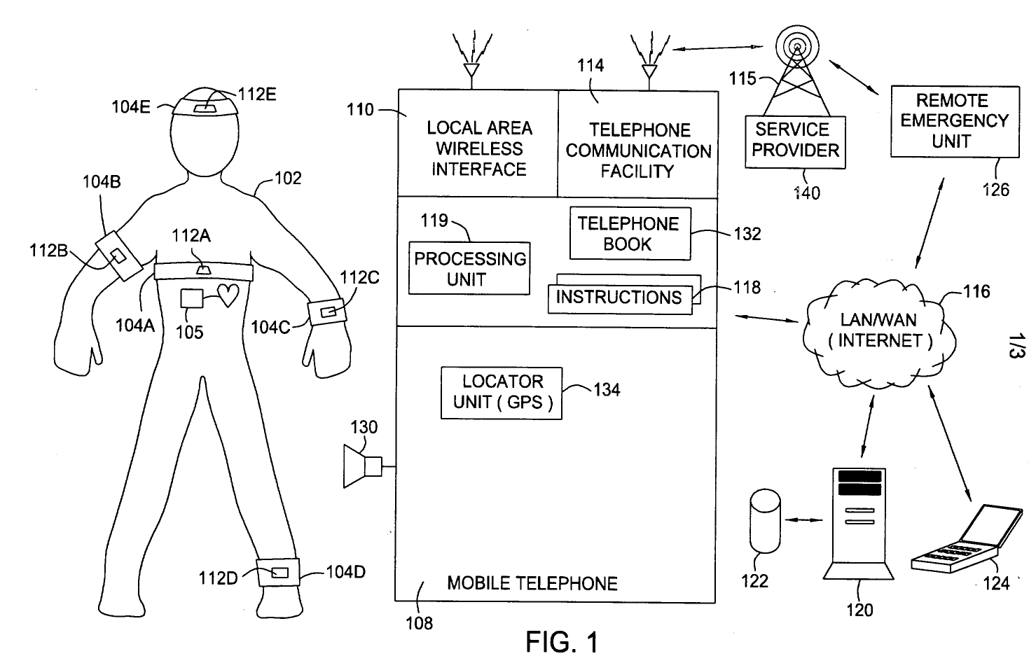

Reference will now be made to the drawings wherein like numerals refer to like parts throughout. Referring to FIG. 1, a human subject (also referred to herein as the “patient”) 102 is shown wearing a plurality of body-worn devices 104A-E (collectively, “body-worn devices 104”, or simply “devices 104”). In general, each body-worn device 104 is configured to perform some medical or health-related function. Illustrative devices include a heart rate monitor 104A, a glucose monitor 104B, a locator unit 104C, a pedometer 104D and a neural monitor 104E. It is understood that these devices are merely illustrative and that any other monitors, known or unknown, are within the scope of the invention. Further, a device 104 may be configured to dispense medicine, such as insulin, according to some predefined schedule or a detected condition of the patient 102. In another embodiment, a device 104 may be configured to implement organ regulation functions by communicating with implanted devices. For example, a device 104 may communicate with an implanted defibrillator 105 which, in turn, operates to produce therapeutic electrical stimulation, e.g., pacing pulses or cardioversion or defibrillator waveforms to stimulate the patient's heart.

In addition to performing a medical/health function, each device 104 is configured to communicate with a wireless unit 108. The communications may be selective (i.e., dependent upon some condition or conditions) or continuous. In the case of selective communications the condition(s) prompting a communication with the wireless unit 108 may be user input from the patient 102 (e.g., pushing a transmission button) or may be a physiological condition detected by a device 104.

The wireless unit 108 is configured with a local area wireless interface 110. As such, each of the devices 104 is configured with a corresponding local area wireless interface 112A-E (collectively local area wireless interfaces 112). The local area wireless interfaces 110, 112 may be, for example, Bluetooth devices or any other of the IEEE 802.11 series wireless networking protocols or Ultra Wide Band wireless technology. Other wireless telemetry devices include those from Zigbee and iBeam. Bluetooth includes the capability of identifying each type of device as it establishes a link to other devices. Thus, a heart rate monitor 104A that has a Bluetooth interface 112A will identify itself as a heart rate monitor. Equipping the wireless unit 108 with a Bluetooth interface 110 allows the wireless unit 108 to automatically detect when it comes in range of one of the medical/health Bluetooth interfaces 112, and vice versa. Other wireless technologies deploying service discovery protocols, such as UDDI, may also be used. Successful communications between the interfaces 110, 112 depend on their relative close proximity. As defined herein, “close proximity” means any distance at which the interfaces 110, 112 are capable of communicating with one another. Persons skilled in the art will recognize that a particular effective distance is dependent upon the technical specifications (e.g., transmission power, signal strength, susceptibility to interference, etc.) of the interfaces 110, 112. In some embodiments, the effective distance between the interfaces 110, 112 may be increased by the provision of intermediary devices capable of augmenting or boosting the transmission signal.

In one embodiment, the wireless unit 108 is configured for communication only with specified devices 104. To this end, the interfaces 110, 112 may each be assigned unique identifiers (e.g., model, type and serial numbers). The identifiers may be included in each transmission to allow the receiving interface to determine whether the transmission should be accepted. Devices 104 may also be distinguished from one another by their operating frequencies, where such frequencies are unique. In any case, the ability to discriminate between devices 104 may be useful, or even necessary, to restrict communication with the wireless unit 108 to only those devices worn by the human subject 102. In this way, traveling with the wireless unit 108 will not result in indiscriminate connections being made with all devices in range of the wireless unit 108.

However, it may be desirable in some cases to allow the wireless unit 108 to receive all signals of a specified type. For example, a particularly advantageous use of the wireless unit 108 is for initiating a distress call in response to a distress signal issued by one of the devices 104. A particular example is where the heart rate monitor 104A detects cardiac arrest of the human subject 102 and issues a distress signal. If the wireless unit 108 receiving the distress call rejects the call as originating from an unrecognized source, the human subject 102 may not receive needed medical attention. As such, it is contemplated that the wireless unit 108 be configured to receive all emergency/distress signals. In this way, a plurality of wireless units 108 act as a network of access points for a given device 104 issuing a distress signal.

In a particular embodiment, the wireless unit 108 is a mobile telephone (and hence, will hereinafter be referred to as the mobile telephone 108). Accordingly, the mobile telephone 108 is equipped with a telephone communication facility 114 capable of communicating through a telephone network 115. The telephone communication facility 114, and the telephone network 115, may be configured for any variety of mobile telephone protocols such as, for example, GSM, GPRS, CDMA; including GSM's short messaging service (SMS), direct Internet access via GSM's GRPS technology or CDMA's data service or even conventional voice services provided by any mobile phone network.

The mobile telephone 108 may also be configured for communication with other devices through, for example, a computer network 116. The computer network 116 may be, for example, a LAN or WAN. In a particular example, the computer network 116 is the Internet. The mobile telephone 108 may communicate with the network 116 through the local wireless interface 110 or by some other communication means, such as a cabled connection. The network connections between the mobile telephone 108 and the networks 115, 116 may be established in response to receiving signals from a device 104, or may be “always on” network connections.

It is noted that the mobile telephone 108 may be configured with additional functionality not shown in FIG. 1. For example, the mobile telephone 108 may also be a personal digital assistant (PDA) having well-known features such as calendars, memo pads, calculators, digital imaging equipment (e.g., a digital camera).

In order to implement one or more functions in response to signals from the devices 104, the mobile telephone 108 is configured with a plurality of instructions 118. In one embodiment, separate instructions are provided for each different device 104. Upon detecting the presence of a particular device 104 through the local wireless interface 110, the appropriate instructions corresponding to that device 104 may be retrieved and executed by a processing unit 119. In addition to having separate instructions for each device 104, a device-specific instruction set may itself include multiple routines selected and executed according to the particular input received from the device 104.

To illustrate the different functions performed by the mobile telephone 108 for a particular device 104, assume that the heart rate monitor 104A is configured to operate in various modes. In a first mode the heart rate monitor 104A may transmit a stream of heart rate data to the mobile telephone 108 which, in turn, logs the heart rate data on a storage device 122 via a network connection via the network 116. In a second mode, the heart rate monitor 104A may transmit a distress signal to the mobile telephone 108 which, in turn, transmits a distress communication over the telephone network 115. In another mode the heart rate monitor 104A may transmit a stream of heart rate data to the mobile telephone 108 which in turn logs the heart rate data on a storage subsystem local to the mobile phone and transmits the data at a later point in time to a server 120 attached to the network 116. In yet another mode the heart rate monitor 104A sends a stream of heart rate data to the mobile telephone 108 which monitors the data and sends a distress signal to the telephone network 115.

From the foregoing examples it should be clear that the mobile telephone 108 is capable of supporting at least one of two functions: data logging and distress calls. In the case of data logging, data received from a device 104 may be logged locally in the memory of the mobile telephone 108, or remotely in a data repository 122 (e.g., a database). Information stored to the remote data repository 122 may subsequently be accessed by the mobile telephone 108 itself or by a computer 124 (e.g., a laptop or PC). The patient may access the data to monitor his/her response to a diet or exercise regiment, for example. Storing patient information remotely in the data repository 122 provides a convenient means of allowing third party access to the data. For example, a physician may access to data repository 122 to facilitate a diagnosis, or to monitor the patient's health. In a particular embodiment, the data is made available through the Internet via a Web server 120.

A distress signal received from a device 104, may prompt the mobile telephone 108 to issue a distress call to a remote emergency unit 126 via the telephone network 115 and/or the computer network 116. In general, signals from the devices 104 may be explicitly configured as distress signals and recognizable by the mobile telephone 108 as such, or may simply be data which the mobile telephone 108 must process to determine whether or not the data indicates an emergency. As an example of the latter, assume that the data is real-time heart rate data for the human subject 102. The mobile telephone 108 may be configured with the necessary programming to determine whether or not the heart rate data indicates an emergency condition (e.g., cardiac arrest or an irregular heart rate). For purposes of the present description, signals transmitted by the devices 104 as explicit distress signals or as signals prompting the mobile telephone 108 to identify an emergency condition are referred to herein as “distress signals”.

In one embodiment, the remote emergency unit 126 is a medical services entity such as a hospital, an ambulance service or a physician. In another embodiment, the remote emergency unit 126 is a family member. In still another embodiment, the remote emergency unit 126 is an intermediary relay station charged with responding to the distress call by, for example, attempting to contact the human subject 102 and, if necessary, contacting an ambulance dispatch service.

It is also contemplated that a single distress signal may result in multiple distress calls being made from the mobile telephone 108. For example, a distress signal from the heart rate monitor 104A indicating cardiac arrest may result in a distress call being made to an ambulance service as well as to a family member living in proximity to the human subject 102. Each entity to be called may have a corresponding telephone number stored in an electronic phone book 132 resident in memory of the mobile telephone 108. In one embodiment, these phone numbers may be stored as a hierarchical list, whereby each number in the list is called in a designated order until the distress call is answered. For example, a first call may be made to a family member of the human subject 102. If the family member does not answer the call, then a second call may be made to the physician of the human subject 102.

The distress call may be in the form of a data signal (i.e., a text messaging signal). In one embodiment, the distress call includes text data which is output on a display of the remote emergency unit 126. The distress call may additionally or alternatively include voice data. In either form, the data contained in the distress call may include the name of the human subject 102 and the particular condition that caused the distress call to be made (e.g., heart failure). In one embodiment, this information may be used to initiate an automated data retrieval process, whereby the patient's medical records are retrieved (from a database) and made available for viewing by a human operator at the remote emergency unit 110. Of course, such a data retrieval process may be manually implemented once the information is received by the human operator.

Preferably, the distress call also includes location information for the human subject 102. To this end, the mobile telephone 108 may include a locator unit 134, such as a Global Positioning System (GPS) unit. Alternatively or additionally, the location information may be provided by the device 104 issuing the distress signal, as in the case were the device 104 is equipped with a GPS device or other locator device. Yet another alternative or additional possibility is that the telephone network 115 provides location information, using mobile phone cell identifications and/or signal triangulation.

The distress call may also include control information which causes a receiving device to perform a designated function, e.g., sound of alarm. For example, a family member of the human subject 102 may be in possession of a mobile telephone configured to sound a distinct alarm upon receiving the distress call from the transmitting mobile telephone 108. In one embodiment, the distress call may include data as well as control information.

In addition to initiating a distress call, the incoming distress signal may also operate to initiate local functions on the mobile telephone 108. For example, the distress signal may cause the sounding of an audible alarm through a speaker 130.

It is contemplated that the distress call function of the mobile telephone 108 may be provided on a fee basis. For example, a telephone service provider 140 may charge a fee (e.g., per usage, on a subscription basis or a one-time fee) to the human subject 102 for the distress call function supported by the mobile telephone 108. For the fee, the telephone service provider 140 may agree to receive calls from mobile telephones initiated in response to signals transmitted from a local area wireless interface of a body monitoring device to a local area wireless interface of the telephone. In one embodiment, the telephone service provider 140 receives the distress calls containing GPS information for device initiating the call. Based on the GPS information, the telephone service provider 140 then contacts (e.g., forwards the distress call) the nearest emergency response entity. In addition, or alternatively, a third party relay station (i.e., the remote emergency unit 126) receiving and handling the distress calls may charge a fee for its services.

Other fee-based applications of the present invention include, for example, drug testing needed for employment purposes, sporting qualifications and the like. Specifically, the human subject 102 may be equipped with a drug testing device configured to provide sample data (e.g., blood analysis data) to the mobile telephone via the local area wireless interface 112 disposed on the human subject 102. It is also contemplated that the drug test may be initiated by a communication from the mobile telephone 108. That is, a control signal is transmitted from the mobile telephone 108 to the testing device, which configures the device to perform the analysis.

Yet another service is the collection and monitoring of health data for clinical and pre-clinical pharmaceutical and other trials providing an automated and continuous data trace for certification and allowing the pharmaceutical company a much quicker time to market. For this service the sensor data can be electronically signed by using, for example, a smart card in the mobile phone, such as a Java SIM card for GSM mobile telephones.

In another application, selective monitoring of the human subject 102 is performed for health insurance purposes. That is, a health insurance company may offer lower premiums to clients willing to wear a body-worn device(s) 104 configured to periodically (or in response to detected condition) provide monitoring data to the health insurance company, or some third-party health services entity responsible for performing additional analysis of the data. A fee may be charged by the health insurance company to the client, and/or by the third-party health services entity to the health insurance company and/or the client.

In yet another application hospital or other health services entities may charge a fee to out-patients to monitor the patients' condition after an operation or diagnosis. By having a constant stream of health care data available, the health care service provider can alert the patient when the patient has forgotten to take a measurement (for example, a blood sugar reading in the morning), can call in the patient for consultation when a deterioration of her health is detected and prevent, for example, hospitalization by early medication. In this manner, health services entities are also able to reduce the relatively higher costs associated with in-patient care while simultaneously giving patients the freedom to leave the hospital.

Another service envisioned is an exercise and training service that allows a customer to keep track of her training/exercise progress via a personalized web portal page in the Internet, the service provider can then provide additional training recommendations based on the data received from the health devices.

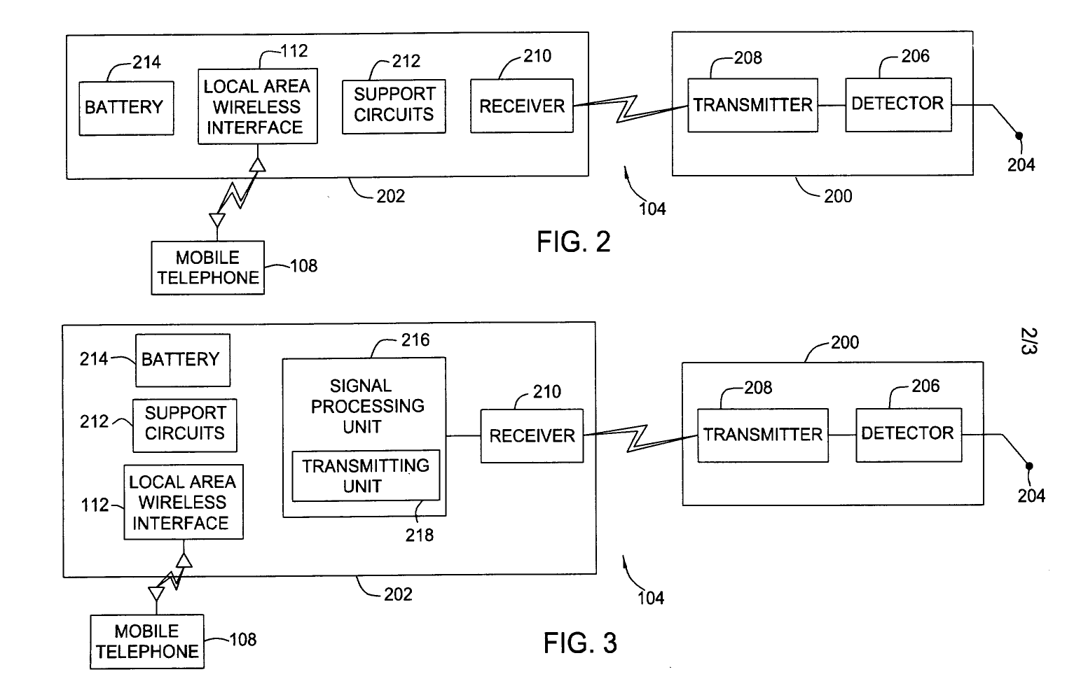

Referring now to FIG. 2, a functional block diagram of one embodiment of a body-worn device 104 is shown. Generally, the device 104 includes a detection unit 200 and a relay device 202. Although not shown, the unit 200 and device 202 may be equipped with, or disposed on, harness or support elements in order to be worn on the human subject 102.

Generally, the detection unit 200 includes a sensor 204 configured to sense, for example, a heart rate, brainwave activity, glucose levels, movement/acceleration (as in the case of an accelerometer), or other activity associated with the human subject 102. In response to selected input, the sensor 204 produces a signal detected by a detector 206. The output of the detector 206 is provided to a transmitter 208, which operates to transmit information to a receiver 210 of the relay unit 202.

In addition to the receiver 210, the relay unit 202 may include support circuits 212, a power source such as a battery 214, and the local area wireless interface 112. Illustrative support circuits include voltage regulators, converters, a recharge facility, one or more processors, memory and the like.

In one form or another, information received by the receiver 210 is provided to the local area wireless interface 112, which communicates information to the mobile telephone 108. As noted above, local area wireless interface 112 maybe any short-range communications device capable of communicating with the mobile telephone 108. In one embodiment, the local area wireless interface 112 is an RF telemetry device, and in a particular embodiment, is a Bluetooth transmitter.

In some embodiment, information received from the detection unit 200 may be substantially unprocessed before being transmitted to the mobile telephone 108. In another embodiment, information may undergo some degree of processing before being transmitted. To this end, the relay device 202 may be equipped with a signal processing unit 216, as shown in FIG. 3. The signal processing unit 216 may perform a variety of functions including, for example, a time stamping function implemented by timestamp unit 218. Time stamping information received from the detection unit 200 may be useful, for example, where communication between the relay device 202 in the mobile telephone 108 is periodically lost. Where the communication link is subsequently re-established, it can be determined based on the timestamps which information has not yet been transmitted to the mobile telephone 108. In one embodiment, the mobile telephone 108 gathers and timestamps signals from one or more of the portable, externally worn body monitoring or relay devices 104 and periodically transmits a collection of signals at regular intervals over the mobile telephone's data channel to a system running in the network.

The signal processing unit 216 may also be used to identify an emergency condition. For example, in the case of a heart rate monitor 104A (FIG. 1) the signal processing unit 216 may determine a cardiac arrest. In response, a distress signal may be issued to the mobile telephone 108 via the local area wireless interface 112. In one embodiment, the signal processing unit 216 may continually process data received from the detector unit 200 and provide the data to local area wireless interface 112 until a cardiac arrest is determined, at which time the distress signal is dispatched. Alternatively, the signal processing unit 216 may be configured to selectively communicate with the local wireless interface 112, i.e., only in the event that an emergency condition is detected.

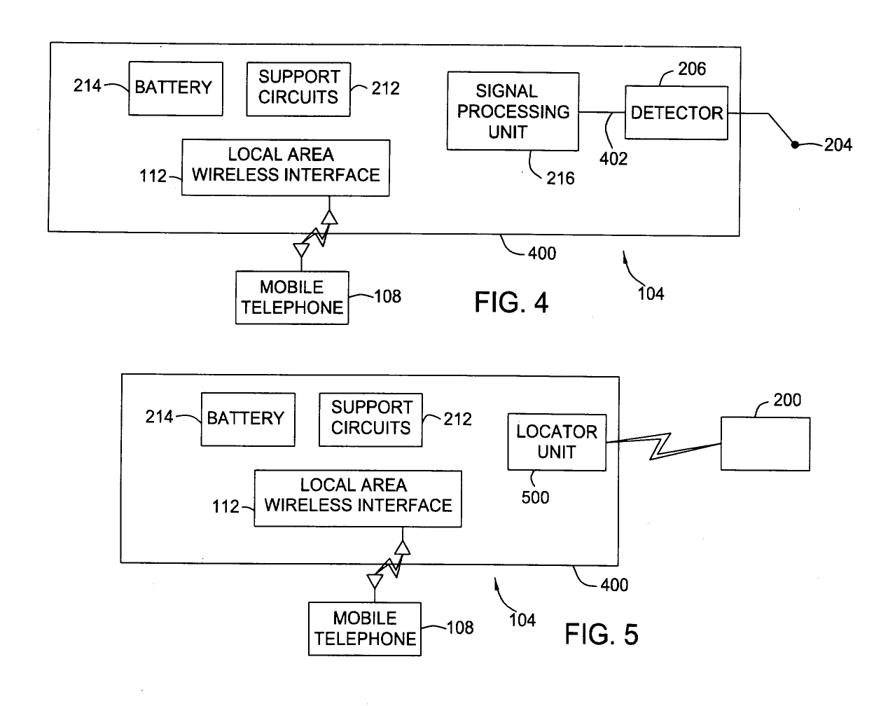

Referring now to FIG. 4, an integrated embodiment of the body-worn device 104 shown, such as where the device is described above are formed on the single printed circuit board (PCB). In this case, the detector 206 may communicate directly with elements of the relay device 202, such as a signal processing unit 216, via a hardwired signal path 402.

FIG. 5 shows yet another embodiment in which the body-worn device 104 is equipped with a locator unit 500. The locator unit 500 may be, for example, a GPS unit. Although not shown, the device 104 may also include the sensor 204 and detector 206 described above. In any case, the locator unit 500 is used to provide information data (specifying the location of the device 104) to the local area wireless interface 112. In this way, the location of the human subject 102 may be provided to the recipient of the distress call may through the mobile telephone 108. As noted above, the mobile telephone may also, or alternatively, be equipped with a locator unit 134. In either case, the provision of locator units may also be used to determine whether the human subject has exceeded the boundaries of a permitted area. For example, the human subject may be a mentally ill person who must be confined to a defined geographic area. If the human subject ventures outside of the defined geographic area (which may be determined by a signal processing unit on board the device 104, for example, using the location information provided by the locator unit 500), an authority (e.g., a nurse) may be notified.

In each of the foregoing embodiments, the body-worn device 104 may cooperate with other devices, such as an implanted device. In a particular embodiment, the body-worn device 104 cooperates with an implanted defibrillator. A distress signal then may be transmitted to the mobile telephone 108 only after the system 100 has attempted to stabilize the heart of the human subject 102 (such as by providing electrical signals to the heart via a pulse generator of the implanted device) a threshold number of times within a predetermined time period. Further, the distress signals may be transmitted to the mobile telephone 108 even in cases where the heart of the human subject 102 is operating normally. For example, the power level of the implanted device may be dangerously low. The low battery power may trigger the body-worn device 104 to transmit a distress signal to the mobile telephone 108.

While the foregoing is directed to embodiments of the present invention, other and further embodiments of the invention may be devised without departing from the basic scope thereof, and the scope thereof is determined by the claims that follow.

What is claimed is:

1. A method for placing a distress call from a mobile telephone in response to detected body functions of a human subject, comprising:
receiving, by a local area wireless interface of the mobile telephone, a wireless signal from a corresponding local area wireless interface disposed on an external body-worn device;
in response to receiving the wireless signal, dialing a telephone number corresponding to a device; and
transmitting, from a wireless telephone communications facility of the mobile telephone, the distress call to the device.

2. The method of claim 1, further comprising transmitting another distress call to another device in the event the device does not receive the distress call.

3. The method of claim 1, further comprising sequentially transmitting the distress call to a plurality of devices according to a predefined order.

4. The method of claim 1, wherein the wireless signal is indicative of a medical emergency experienced by the human subject wearing the body-worn device.

5. The method of claim 1, wherein receiving the wireless signal occurs only when the body-worn device determines that the human subject wearing the body-worn device is experiencing an emergency condition.

6. The method of claim 1, prior to transmitting, determining that the wireless signal is indicative of a medical emergency being experienced by the human subject wearing the body-worn device.

7. The method of claim 1, wherein the wireless signal and distress call contain data pertaining to an organ being monitored by a medical device disposed on the human subject.

8. The method of claim 1, wherein the distress call contains location information indicating a location of the body-worn device.

9. A wireless telephone for selectively placing a telephone call, comprising:
a local area wireless interface configured to receive transmissions from a corresponding local area wireless interface disposed on a body-worn device externally disposed on a human subject;
a wireless telephone facility;
a processing unit; and
instructions for configuring the processing unit to respond to transmissions from the body-worn device by initiating the telephone call, via the wireless telephone facility, to a predefined telephone number.

10. The wireless telephone of claim 9, wherein the local area wireless interface is a Bluetooth transmitter.

11. The wireless telephone of claim 9, wherein the processing unit is configured by the instructions to add location information to the telephone call, the location information indicating a location of the body-worn device.

12. The wireless telephone of claim 9, further comprising a locator device configured to include location information to information transmitted during the telephone call.

13. The wireless telephone of claim 9, wherein the processing unit is configured by the instructions to add to the telephone call data pertaining to an organ being monitored by a monitoring device disposed in the human subject.

14. The wireless telephone of claim 9, wherein the transmission is indicative of a medical emergency experienced by the human subject wearing the body-worn device and configures the processing unit provide information via the telephone call to indicate the emergency.

15. An external body-worn relay device, comprising:
a receiver configured for wireless communication with a body sensor;
a body monitoring device configured to receive signals received from the body sensor via the receiver and monitor an aspect of human activity of a human subject; and
a local area wireless transmitter in communication with the monitoring device and configured to transmit a wireless signal in response to a local area wireless interface of a mobile telephone.

16. The external body-worn relay device of claim 15, wherein the local area wireless transmitter is configured to transmit the wireless signal in response to the monitoring device determining a condition of the human subject wearing the sensor.

17. The external body-worn relay device of claim 15, wherein the monitoring device comprises an organ monitoring device configured to monitor activity of an organ, the activity being sensed by the sensor.

18. The external body-worn relay device of claim 17, wherein the local area wireless transmitter is activated by the organ monitoring device to transmit the wireless signal only when the organ monitoring device determines that detected organ activity indicates the organ is experiencing an emergency condition.

19. The external body-worn relay device of claim 18, wherein the wireless signal transmitted in response to detecting organ activity indicates the organ is experiencing the emergency condition configures the mobile telephone to initiate a distress call to a predefined telephone number.

20. The external body-worn relay device of claim 15, wherein the local area wireless transmitter is a Bluetooth transmitter.

21. The external body-worn relay device of claim 15, further comprising a locator device configured to determine a location of the external body-worn relay device.

22. A portable externally worn body monitoring device, comprising:
a monitoring device configured to monitor an aspect of human activity of a human subject;
a local area wireless transmitter in communication with the monitoring device and configured to transmit a wireless signal to a local area wireless interface of a mobile telephone; and
a support member for supporting the monitoring device and the local area wireless transmitter on the human subject.

23. The device of claim 22, wherein the local area wireless transmitter is a Bluetooth transmitter.

24. The device of claim 22, further comprising a locator device configured to determine a location of the portable externally worn body monitoring device.

25. The device of claim 22, wherein the local area wireless transmitter is configured to transmit the wireless signal in response to the monitoring device determining a condition of the human subject.

26. The device of claim 22, wherein the monitoring device comprises an organ monitoring device configured to monitor activity of an organ.

27. The device of claim 26, wherein the local area wireless transmitter is activated by the organ monitoring device to transmit the wireless signal only when the organ monitoring device determines that detected organ activity indicates the organ is experiencing an emergency condition.

28. The device of claim 27, wherein the wireless signal transmitted in response to detecting organ activity indicating the organ is experiencing the emergency condition configures the mobile telephone to initiate a distress call to a predefined telephone number.

29. A portable externally worn body monitoring device, comprising:
a monitoring device interface configured to interface with a monitoring device monitoring an aspect of human activity of a human subject;
a local area wireless transmitter in communication with the monitoring device and configured to transmit a wireless signal to a local area wireless interface of a mobile telephone; and
a support member for supporting the monitoring device and the local area wireless transmitter on the human subject.

30. The device of claim 29, wherein the local area wireless transmitter is a Bluetooth transmitter.

31. The device of claim 29, further comprising a locator device configured to determine a location of the portable externally worn body monitoring device.

32. The device of claim 29, wherein the local area wireless transmitter is configured to transmit the wireless signal in response to the monitoring device determining a condition of the human subject.

33. The device of claim 29, wherein the monitoring device comprises an organ monitoring device configured to monitor activity of an organ.

34. The device of claim 33, wherein the local area wireless transmitter is activated by the organ monitoring device to transmit the wireless signal only when the organ monitoring device determines that detected organ activity indicates the organ is experiencing an emergency condition.

35. The device of claim 34, wherein the wireless signal transmitted in response to detecting organ activity indicating the organ is experiencing the emergency condition configures the mobile telephone to initiate a distress call to a predefined telephone number.

36. A method of providing a distress call service by a mobile telephone service provider, comprising:
receiving distress calls from mobile telephones equipped with local area wireless interfaces configured to communicate with corresponding local area wireless interfaces of portable externally worn body monitoring devices, wherein the portable externally worn body monitoring devices cause the mobile telephones to transmit the distress calls by transmitting a signal from their respective corresponding local area wireless interfaces to the local area wireless interfaces of the wireless telephones;
connecting the mobile telephones to receiving devices having telephone numbers to which the distress calls are made;
transmitting, from the mobile telephone, information indicating an emergency to the receiving devices; and
charging a fee for the connecting and transmitting.

37. The method of claim 36, wherein the information transmitted to the devices includes location information indication a location of one of the respective mobile telephones and the portable externally worn body monitoring devices.

38. The method of claim 36, wherein the local area wireless interfaces are Bluetooth transmitters.
